(19) United States
(12) Patent Application Publication
(10) Pub. No.: US 2006/0235308 A1
(43) Pub. Date: Oct. 19, 2006

(54) System for dark-field imaging of target areas below an object surface

(76) Inventors: Michael Cornelis Van Beek, Eindhoven (NL); Frank Jeroen Pieter Schuurmans, Eindhoven (NL)

Correspondence Address: PHILIPS INTELLECTUAL PROPERTY & STANDARDS, 595 MINER ROAD, CLEVELAND, OH, 44143, US

(21) Appl. No.: 10/567,284

(22) PCT Filed: Jul. 29, 2004
(86) PCT No.: PCT/IB04/51327

§ 371 Date: Feb. 3, 2006

(30) Foreign Application Priority Data
Aug. 8, 2003 (EP) 03102481.3

(51) Int. Cl. A61B 6/00; A61B 5/00

(52) U.S. Cl. 600/473; 600/310

(57) ABSTRACT

A monitoring system for dark-field imaging of a target area (72) below a surface (70) of an object, e.g., capillary vessel under the surface of the skin of a patient, comprises an illumination optical system (31), an imaging system (35), and a selective optical interception system for suppressing illumination light returning from the region between the surface of the object and the target area. The interception system may comprise crossed polarizers (32, 37) in the illumination and imagining paths, respectively, and/or an aperture stop (51) arranged to intercept a central portion of the returning imaging beam. Alternatively, the illumination and imaging paths subtend an angle and the illumination focus and the imaging focus are displaced relative to each other. The monitoring system may be comprised in an analysis apparatus further comprising a spectroscopy system having an excitation system and a detection system.

The invention pertains to an analysis apparatus comprising a spectroscopy system that includes an excitation system to emit an excitation beam to a target area below a surface of an object and the analysis apparatus further comprising a monitoring system to image the target area

Such an analysis apparatus is known from the international application WO02/057759.

The known analysis apparatus employs the monitoring system to monitor the target region while the target region is excited by the excitation beam. On the basis of an image formed of the target region, the excitation beam is accurately aimed at the target region. Hence, the scattered radiation is essentially generated in the target region, so that the scattered radiation that is detected includes information that essentially pertains to the material composition in the target region. In order to image the target area when that target area is located under the surface of the object the known system is fitted with a scanning confocal optical imaging system. Notably, the target area may be a capillary blood vessel that is located under the surface of the skin of a patient to be examined. The known analysis apparatus includes a complex and expensive confocal optical system to image the target area and receive scattered radiation from essentially only the target area below the surface of the object.

An object of the invention is to provide an analysis apparatus with a monitoring system to image the target area below the surface of the object which is simpler and cheaper to manufacture.

This object is achieved by an analysis apparatus in which according to the invention the monitoring system includes
a illumination optical system to emit an illumination beam along an illumination beam path onto the object and
an imaging system to receive a returning imaging beam from the target area along an imaging beam path, wherein
the illumination beam path and the imaging beam path subtend an angle.

The monitoring system illuminates the target area by way of effective back-illumination. The illumination beam penetrates into the object and is scattered within the object so as to cause back-illumination of the target area. Substantially, back-illumination is performed by way of multiply scattered diffused and depolarised radiation which is spatially quite uniformly distributed. Accordingly, an even back-illumination of the target area is achieved. Illumination is performed with (electromagnetic) radiation in the range between ultraviolet radiation, visible light to infrared radiation. Preferably, yellow/green radiation in the range of 520 to 580 nm is employed. Because the target area is mainly illuminated by scattered radiation, dark-field imaging is achieved. Because there is hardly any contribution to the imaging from the specularly reflected illumination, the target area is imaged at a high contrast.

For example, such back-illumination is achieved by way of orthogonal polarised spectral imaging, which involves a polarised illumination beam and employing a polarisation-analyser having its polarisation-axis transverse to the polarisation direction of the illumination beam, i.e. in short to employ crossed-polarisers in the illumination beam and the imaging beam respectively. The scattered radiation that has illuminated the target area and returns from the target area is collected by the imaging optical system. In this way, according to the invention, back-illumination is achieved by means of relatively few simple optical components that need not satisfy very accurate specification. Hence, the monitoring system of the analysis apparatus of the invention is simple and inexpensive to manufacture. Because the illumination beam path and the imaging beam path do not coincide, essentially the entire numerical aperture of the imaging optical system is available for imaging, so that the effective optical sensitivity of the imaging optical system is improved. Also, the illumination of the target area is made more efficient because the illumination beam does not need to pass through optical elements of the imaging optical system. Further, as the illumination beam path and the imaging beam path are not coincident, the illumination optical system and the imaging optical system can to a large degree be independently optimised. That is, in the analysis apparatus of the invention, the illumination optical system and the imaging optical system can be independently designed. Also, the imaging optical system operates more efficiently because the returning scattered radiation that the imaging optical system uses to image the target area does not pass through optical elements of the illumination optical system. Further, the analysis apparatus of the invention is able to perform orthogonal polarised spectral imaging of the target area with a good contrast resolution because the crossed-polarisers are placed close to the object to be examined, so that unintentional depolarisation by optical elements is avoided.

The analysis apparatus is further provided with a detection system to receive scattered radiation from the target area The scattered radiation is generated by excitation of the target area by the excitation beam which is produced by the excitation system. For example Raman scattering occurs in the target area due to optical excitation. Preferably, the detection system is able to resolve the components of respective wavelength ranges of the radiation that is scattered from the target area, i.e. to perform a spectroscopic analysis of the target area The relative contributions in these respective wavelength ranges provide useful information about the composition of matter in the target area.

The analysis apparatus of the invention is advantageously employed to investigate in vivo blood non-invasively. The, object to be examined is a patient to be examined and the target area concerns for example a capillary blood vessel under the surface of the patient's skin. The monitoring system images the capillary blood vessel in the patients upper layer of the skin so that the excitation beam can be accurately directed to the capillary blood vessel. Notably, back-illumination is achieved through scattering from the tissue structures in the deeper layers of the skin. Because the excitation beam is accurately directed to the target area, i.e. the capillary vessel, scattering, such as Raman scattering, is generated mainly from the capillary blood vessel and hardly in the surrounding skin tissue. Accordingly, the detection system receives essentially only (Raman) scattered radiation from the target area and contamination of the spectroscopic analysis of the target area by signals from its surrounding is avoided.

It is noted that a monitoring system in which the illumination beam path and the imaging beam path subtend an angle is known per se from the web-site www.olympus.com/primer/techniques/darkfield.html, which discloses a reflected dark field objective. However, the known reflected dark field objective is arranged to investigate the surface itself of an object to be examined. The cited website does not teach to employ the known reflected dark field objective for imaging structure below a surface.

The invention also relates to a monitoring system for dark-field imaging of a target below the surface of the object.

The monitoring system according to the invention is provided with a selective optical interception system to intercept returning radiation from the region between the target and the surface. The selective optical interception system achieves that mainly radiation that has propagated from behind the target, as seen from the surface of the object, contribute to the image of the target. Hence, an image having good contrast is formed of the target area.

These and other aspects of the invention will be further elaborated with reference to the embodiments defined in the dependent Claims.

For example the selective optical interception system includes a polariser in the illumination beam path and an analyser in the imaging beam path; the polariser and analyser at crossed polarisation orientations. Then essentially only radiation from the target area that has undergone multiple scattering is received by the imaging system. In particular specularly reflected radiation from the illumination beam will have preserved its polarisation and will be intercepted by the analyser. Radiation that has been multiply scattered in the object will be depolarised to some extent and will be transmitted by the analyser. Multiply scattered radiation will occur mainly from regions below the target area, seen from the surface of the object, while specularly reflection of the illumination beam occurs mainly at the surface of the object and radiation that has undergone only few scatterings and has preserved its polarisation arises mainly from the region between the surface of the object and the target area Hence, the target area is imaged a high contrast.

In another example the selective optical interception system includes an aperture stop that intercepts a central portion of the returning imaging beam. This central portion includes mainly specularly reflected portions and portions having one or very few scatterings. Interception of the central portion of the returning imaging beam causes contributions to the returning imaging beam from the region between the surface and the target area to be suppressed, so that the target area is imaged at high contrast. It is noted that from the international application WO00/27276 it is known to form a ring shaped illumination pattern around the target area within the object to be imaged. Although such a ring shaped illumination pattern avoids contributions to the imaging beam from the region between the surface and the target area, this ring shaped illumination pattern relatively inefficiently illuminates the target area.

In an alternative embodiment the illumination beam produces an unfocused illumination beam. Hence, illumination of the surface and the region between the target area and the surface is relatively reduced relative to the illumination of the target area and the region below the target area. This unfocussed illumination beam preferably covers a wide range around and below the target area so that efficient uniform back-illumination of the target area is achieved.

In an other embodiment the illumination beam and the returning imaging beam are at an angle and have their respective focus mutually displaced. That is the focus position of the illumination beam in the object is displaced from the imaging focus that is sharply imaged by the imaging system. Because the illumination beam is at an angle relative to the path of the imaging optical system, specularly reflection from the surface can hardly or not at all contribute to the imaging beam. Because the focus of the illumination beam is displaced from the focus of the imaging system, directly reflected light from around the target area is avoided in the imaging beam. Notably, the focus of the illumination beam is placed outside of the region between the target area and the surface of the object. Accordingly, the back-illumination of target area can be controlled independently of the acquisition of the imaging beam by the imaging optical system.

Further according to the invention, the monitoring system as defined in any one of Claims 1, to 6 are advantageously employed in an analysis apparatus. The analysis apparatus comprises a spectroscopy system having and excitation system. The excitation system produces an excitation beam that is directed towards the target area to cause (optical) excitation localised to the target area. The monitoring system is employed in the analysis apparatus to image the target area so that the excitation can be accurately directed onto the target area. Because the monitoring system of the invention as defined in any one of claims 1 to 6 produces a high contrast image of the target area, notably when the target area is located below the surface of the object, the analysis apparatus that comprises the monitoring system of the invention can more accurately direct the excitation beam onto the target area and consequently spectroscopic data that are essentially originating form target area can be acquired. Very good results are achieved when the analysis apparatus of the invention is employed to perform Raman spectroscopy in vivo to blood in a capillary blood vessel located below the surface of the skin of the person to be examined. These and other aspects of the invention will be elucidated with reference to the embodiments described hereinafter and with reference to the accompanying drawing wherein

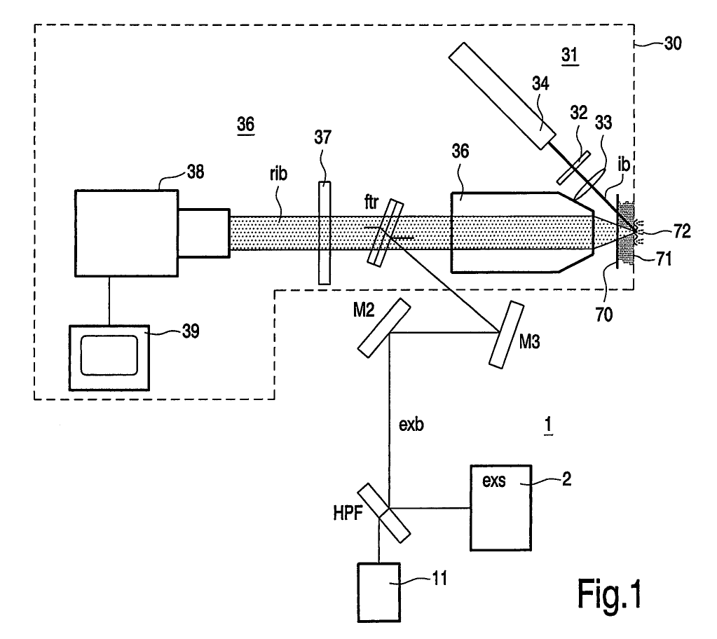

FIG. 1 shows a diagrammatic representation of an example of the analysis apparatus in which the invention is employed;

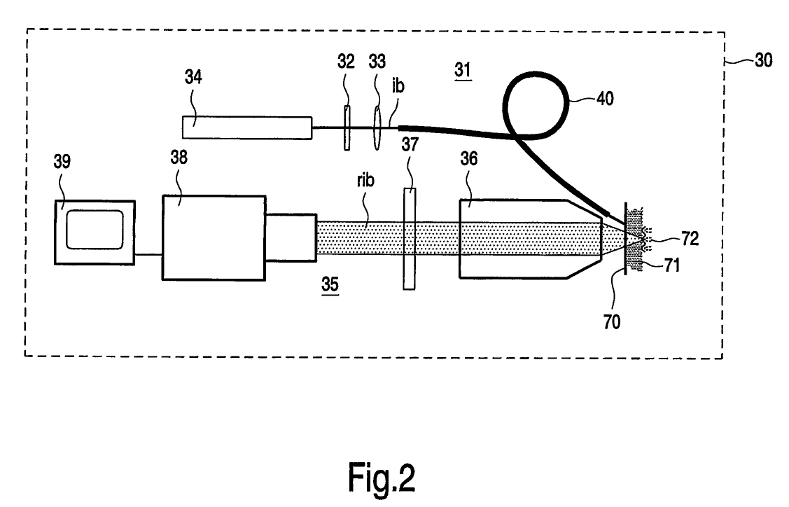

FIG. 2 shows an embodiment of the monitoring system of the invention,

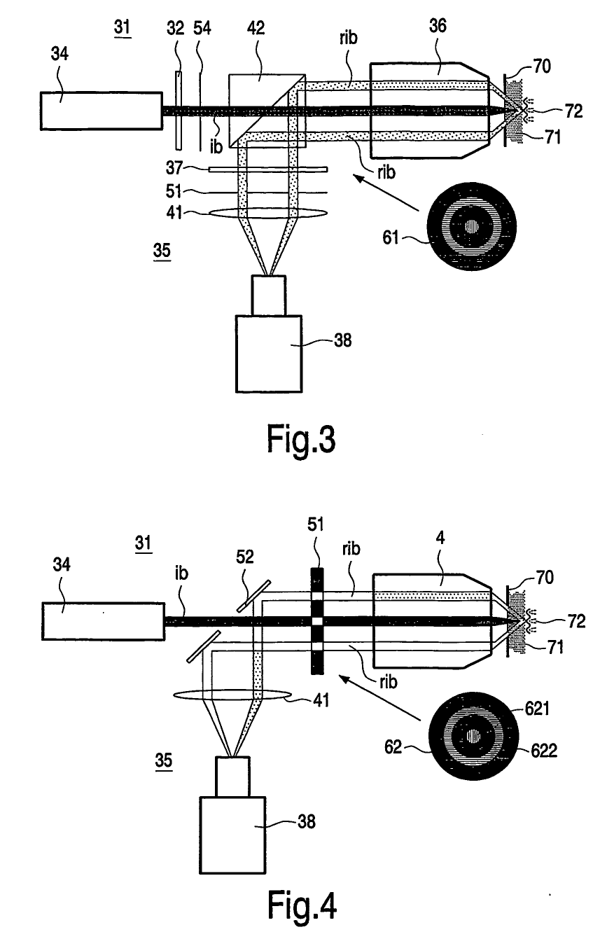

FIG. 3 shows an embodiment of the monitoring system of the invention wherein one version the selective optical interception system is employed and

FIG. 4 shows an embodiment of the monitoring system wherein another version of the selective optical interception system is employed.

FIG. 1 shows a diagrammatic representation of an example of the analysis apparatus in which the invention is employed. The analysis apparatus of the example shown in FIG. 1 is in particular designed for examination of the content of blood in the capillary blood vessels in the patient's skin. In this application the patient to be examined is the object to be examined and the target area 72 is formed by the capillary blood vessel in the patient's skin. The analysis apparatus comprises the monitoring system 30 and a spectroscopy unit 1. The spectroscopy unit 1 includes the excitation system which emits the excitation beam (exb) and the detection system 11 to receive scattered radiation from the target area 72. The detection system 11 is able to resolve the components of respective wavelength ranges to perform a spectroscopic analysis of the target area 72.

The monitoring system images the target area 72. To that end, the monitoring system is provided with the illumination optical system 31 which includes a light source 34, a polariser 32 and a lens 33. The light source 34 is for example a lamp, a laser, preferably a semiconductor laser or solid-state laser, or a light-emitting diode (LED). Preferably, the light source emits green to yellow light having a wavelength in the range 520-580 nm where notably good contrast of blood vessels with respect to the surrounding tissue is achieved. The polariser 32 polarises the light from the light source. The lens 33 focuses the polarised light in the skin of the patient to be examined. Within the skin tissue, notably in the tissue layers below the capillary blood vessels the polarised light is scattered and becomes depolarised. The scattered light back-illuminates the capillary blood vessels which are closer to the skin surface 70.

The scattered light from the patient's skin is employed to form an image of the capillary blood vessels by the imaging optical system 35. To that end the imaging optical system comprises an objective lens 36, an analyser 37, a CCD-camera 38 and a monitor 39. The light that returns from the patient's skin is collected by the objective lens 36, which forms the returning light beam (rib). The optical axis of the imaging optical system, in particular the optical axis of the objective lens is orientated at an angle to the optical axis of the illumination optical system, notably the optical axis of the lens 33. The analyser 37 is orientated such that its polarisation axis is in the crossed orientation with respect the polarisation axis of the polariser 32 in the illumination optical system 31. Accordingly, essentially only light that has become depolarised to a substantial degree in the deeper layers of the patient's skin is passed through the analyser 37 and is employed to form the image of the capillary blood vessels. The image is acquired by the CCD-camera 38 which derives an electronic image signal from the acquired image. The signal levels of the electronic image signal represent the brightness values of the image of the capillary blood vessels. The CCD-camera 38 applies the electronic image signal to the monitor to display the image of the capillary blood vessels or to an image processing system such as a PC for automatic blood vessel detection.

The excitation system 1 includes a laser system 11 to emit the excitation beam. For example, a diode laser is used which emits the excitation beam (exb) having a wavelength in a range around 785 nm. The excitation beam is reflected at the high pass filter HPF and directed towards the objective via the mirrors M2, M3 and dichroic mirror ftr. The inelastically scattered Raman light is transmitted through the high pass filter HPF and directed towards the detection system 11.

FIG. 2 shows an embodiment of the monitoring system of the invention. The imaging optical system of the monitoring system of FIG. 2 is similar to the monitoring system of the analysis apparatus shown in FIG. 1. The illumination system comprises an optical fibre 40 which directs the illumination beam from the lens 33 to the patient's skin. Because the optical fibre 40 is flexible, this allows easy direction of the illumination beam towards the patients skin. In particular, it is easy to displace the focus of the illumination beam from the focus of the imaging optical system. The direction of the illumination beam when it exits the optical fibre 40 makes an angle to the optical axis of the imaging optical system. Alternatively it is possible to place a lens in front of the fibre to produce a focus.

FIG. 3 shows an embodiment of the monitoring system of the invention wherein one version the selective optical interception system is employed. The selective optical interception system shown in FIG. 3 includes the polariser 32 in the illumination beam (ib) and the analyser 37 is placed in the returning imaging beam (rib). Additionally, the selective optical interception system is provided with ring-shaped aperture stop. In the insert a cross section 61 is shown of the imaging beam transmitted to the CCD-camera. The polariser 32 and analyser 37 are at so-called crossed orientations. Hence, only the contribution to the returning imaging beam that arises from multiple depolarising scatterings around the target area 72 below the surface 70 of the object are passed through the analyser to the CCD-camera 38. A polarising beam splitter 42 is employed to pass the polarised illumination beam towards the objective lens 36 to illuminate the target area 72. The polarising beam splitter reflects the returning imaging beam towards the CCD-camera 38. An imaging lens is used to focus the returning imaging beam onto the CCD-sensor of the CCD-camera 38. Further, the aperture stop suppresses returning radiation from the surface 70 of the object and from the region 72 between the surface 70 and the target area 71.

FIG. 4 shows an embodiment of the monitoring system wherein another version of the selective optical interception system is employed. Here the selective optical interception system includes a mirror 52 having a hole in its centre and an aperture stop 51 having an opening in the centre as well as a ring shaped opening in the periphery. In the insert a cross section 62 is shown of the imaging beam transmitted to the CCD-camera. Preferably, the aperture stop has crossed polarisers for illuminating and imaging radiation, that is in the central opening 621 and the peripheral opening 622 polarisers at crossed polarisation orientation are provided. Hence, the central opening 621 transmits and polarises the illumination beam and the peripheral opening 622 suppresses specularly reflected radiation that has preserved its polarisafion and multiply scattered radiation of the returning imaging beam is passed through the peripheral opening. Further, the mirror 52 only passes the peripheral portion to the CCD-camera 38 of the returning imaging beam that concerns multiply scattered radiation. Hence, the aperture stop 51 and the mirror 52 effectively suppress radiation returning from the surface 70 and the region 71 between the target area 72 and the surface 70.

1. A monitoring system for dark-field imaging of a target area below a surface of an object, the monitoring system including
an illumination optical system to emit an illumination beam along an illumination beam path onto the object and
an imaging system to receive a returning imaging beam from the target area along an imaging beam path, wherein
the imaging system includes a selective optical interception system to intercept a returning illumination beam from the region between the surface and the target area.

2. A monitoring system as claimed in claim 1, wherein
the illumination system is arranged to produce the illumination beam as a polarised illumination beam and
the selective optical interception system includes a polarisation-analyser having its axis crossed relative to the polarisation axis of the polarised illumination beam.

3. A monitoring system as claimed in claim 1, wherein the selective optical interception system includes an aperture stop that essentially intercepts a central portion of the returning imaging beam.

4. A monitoring system for dark-field imaging of a target area below a surface of an object, the monitoring system including
an illumination optical system to emit an illumination beam along an illumination beam path onto the object and
an imaging system to receive a returning imaging beam from the target area along an imaging beam path, wherein
the illumination optical system produces an unfocussed illumination beam.

5. A monitoring system for dark-field imaging of a target area below a surface of an object, the monitoring system including
an illumination optical system to emit an illumination beam along an illumination beam path onto the object and
an imaging system to receive a returning imaging beam from the target area along an imaging beam path, wherein
the illumination beam path and the imaging beam path subtend an angle and
the illumination optical system has an illumination focus,
the imaging system has an imaging focus and
the illumination focus being displaced from the imaging focus.

6. An analysis apparatus comprising
a spectroscopy system that includes
an excitation system to emit an excitation beam to a target area below a surface of an object and
the analysis apparatus further comprising a monitoring system to image the target area, the monitoring system including
a illumination optical system to emit an illumination beam along an illumination beam path onto the object and
an imaging system to receive a returning imaging beam from the target area along an imaging beam path, wherein
the illumination beam path and the imaging beam path subtend an angle.

7. An analysis apparatus comprising a spectroscopy system that includes
an excitation system to emit an excitation beam to a target area below a surface of an object and
the analysis apparatus further comprising a monitoring system as claimed in claim 1.
